(19) United States
(12) Patent Application Publication
(10) Pub. No.: US 2021/0393509 A1
(43) Pub. Date: Dec. 23, 2021

(54) AGENT FOR MITIGATING HOT FLASH, COSMETIC PRODUCT, AND METHOD OF USING COSMETIC PRODUCT

(71) Applicant: BLOOM CLASSIC CO., LTD, Tokyo (JP)

(72) Inventors: Hayami TABE, Tokyo (JP); Akihito FUJITA, Tokyo (JP); Mayumi KOTANI, Tokyo (JP); Yasushi TAGANE, Tokyo (JP)

(21) Appl. No.: 17/337,134

(22) Filed: Jun. 2, 2021

(30) Foreign Application Priority Data
Jun. 17, 2020 (JP) 2020-104685

(51) Int. Cl. A61K 8/9789; A61K 8/04

(52) U.S. Cl. CPC A61K 8/9789; A61K 2800/884; A61K 8/042

(57) ABSTRACT

Disclosed is a hot flash-mitigating agent that is highly effective in mitigating hot flashes associated with premenstrual syndrome. The hot flash-mitigating agent contains mallow extract as an active ingredient.

### CROSS REFERENCE TO RELATED APPLICATION

The present application claims priority to Japanese Patent Application No. 2020-104685, filed on Jun. 17, 2020, the entire contents of which are incorporated herein for all purposes by this reference.

### BACKGROUND OF THE INVENTION

### 1. Field of the Invention

The present invention relates to an agent for mitigating hot flashes associated with premenstrual syndrome (PMS). In addition, the present invention relates to a cosmetic product used to mitigate hot flashes associated with premenstrual syndrome (PMS) and to a method of using the cosmetic product.

### 2. Description of the Related Art

A female body repeats a certain cycle due to hormone balance. When this hormone balance changes, various physical symptoms occur due to premenstrual syndrome (PMS). Due to the premenstrual syndrome (PMS), before the start of a menstrual period, the skin secrets more oil (sebum), becomes dull due to dryness, becomes blotchy, and experiences a hot flash, etc. In fact, skin problems that occur during that period are various skin-related symptoms such as oily skin, rough skin, dull skin, a hot flash, etc. In addition, since the skin becomes very sensitive during the premenstrual period, some women feel itchiness.

It is thought that a hot flash is attributable to a change in hormone balance that affects the function of the parasympathetic vasodilator fibers. A hot flash involves various symptoms such as facial flushing, sweating, the feeling of burning and flushing over the face even with the lower body being cold, the feeling of cold, etc.

Conventionally, there are cosmetic products suitable for the skin before or during a menstrual period. Specifically, those conventional cosmetic products are suitable for application to oily skin attributable to increased secretion of sebum, rough skin attributable to dryness, dull skin due to stains worsened, itching skin due to increased sensitivity, etc. However, there is no cosmetic product suitable for skin suffering a hot flash.

### LITERATURE OF RELATED ART

### Patent Literature

[Patent Literature 1] Japanese Patent No. 3390868

### SUMMARY OF THE INVENTION

An objective of the present invention is to provide an agent for mitigating hot flashes associated with premenstrual syndrome (PMS).

To accomplish the objective, a first invention provides an agent for mitigating hot flashes associated with premenstrual syndrome (PMS), the agent containing mallow extract as an active ingredient (Invention 1).

A second embodiment provides a cosmetic product used to mitigate hot flashes associated with premenstrual syndrome (PMS), the cosmetic product being prepared by blending mallow extract (Invention 2).

In the second invention (Invention 2), the cosmetic product includes a first cosmetic product that is an essence phase blended with mallow extract and a second cosmetic product that is a gel phase blended with mallow extract, in which the first cosmetic product and the second cosmetic product are contained in respectively different containers (Invention 3).

In the third invention (Invention 3), it is preferable that the first cosmetic product and the second cosmetic product are sequentially used in this order (Invention 4).

The present invention uses mallow extract as an active ingredient, thereby providing an agent for highly effectively mitigating hot flashes associated with premenstrual syndrome (PMS).

### DETAILED DESCRIPTION OF THE DISCLOSURE

Hereinafter, embodiments of the invention will be described.

An “agent for mitigating hot flashes associated with premenstrual syndrome (PMS)” (hereinafter, simply referred to as “hot flash-mitigating agent”), according to the invention, uses mallow extract as an active ingredient.

In the embodiments of the invention, the term “extract” refers to an extract that is extracted from mallow plants that serve as an extract source, a diluent or concentrate of the extract, and a dried product that is obtained by drying the extract, or a crude or purified form of each of those extracts.

Malva mauritiana L is a perennial herb belonging to the mallow genus Malva in the family of Malvaceae, which origin is southern Europe. The Malva mauritiana L is commonly known as common mallow and is cultivated as an herbal plant in Japan. For example, the constituent parts of a common mallow plant, which can be used as raw materials for mallow extract, are above-ground parts such as flowers, leaves, and stems, underground parts such as roots, or combinations of the above-ground parts and the under-ground parts. However, flowers, leaves, and stems are preferably used to obtain mallow extract.

The mallow extract can be obtained by drying the raw materials (Malva mauritiana L plants), pulverizing the dried raw materials, and immersing the pulverized raw materials in an extraction solvent. Alternatively, the pulverization may be optionally performed. The drying may be carried out in the sun or with a dryer that is commonly used.

As the extraction solvent, it is preferable to use one or more polar solvents such as water and hydrophilic organic solvents. Each of those polar solvents may be used alone or in combination with others at room temperature or below the boiling point of the selected polar solvent.

Examples of water that can be used as the extraction solvent include pure water, tap water, well water, mineral spring water, mineral water, hot spring water, spring water, and fresh water, and treated water obtained through various water treatment processes. For example, the water treatment processes that may be used to produce the treated water include purification, heating, sterilization, filtration, ion exchange, osmotic pressure adjustment, and buffering. Therefore, in the present embodiment, examples of the water that can be used as the extraction solvent include purified water, heated water, ion exchanged water, saline, phosphate buffer, phosphate buffered saline, etc.

Examples of the hydrophilic organic solvent that can be used as the extraction solvent include: lower aliphatic alcohols having 1 to 5 carbon atoms, such as methanol, ethanol, propyl alcohol, and isopropyl alcohol; lower aliphatic ketones such as acetone and methyl ethyl ketone; and multivalent alcohols having 2 to 5 carbon atoms, such as 1,3-butylene glycol, propylene glycol, and glycerin.

When a mixture of two or more polar solvents is used as the extraction solvent, the mixing ratio of the polar solvents is suitably adjusted. For example, when a mixture of water and a lower aliphatic alcohol is used as the extraction solvent, the mixing ratio of the water and the lower aliphatic alcohol is preferably in a range of 9:1 to 1:9 (based on volume) and more preferably in a range of 7:3 to 2:8 (based on volume). On the other hand, when a mixture of water and a lower aliphatic ketone is used, the mixing ratio thereof is preferably in a range of 9:1 to 2:8 (based on volume). When a mixture of water and a multivalent alcohol is used, the mixing ratio is preferably in a range of 8:2 to 1:9 (based on volume).

The extraction process is not particularly limited if soluble components in the extraction raw material can be eluted in the extraction solvent. The extraction may be performed according to any conventional method. For example, an extract can be obtained through the steps of: immersing the extraction raw material in an extraction solvent that is in an amount of 50 to 250 times (by weight) the extraction raw material; extracting soluble components at room temperature or under reflux heating; and filtering the resulting solution to remove the residue. Distilling off the solvent from the filtered solution gives a paste-like concentrate, and drying the concentrate gives a dried product of the extract.

The extract obtained through the method described above can be used as it is as an active ingredient of the hot flash-mitigating agent. In some cases, the extract can be more easily used when it is provided in the form of a concentrate or a dried product.

Since the mallow extract obtained through the method described above has an excellent effect on improving hot flashes associated with premenstrual syndrome (PMS), the mallow extract can be used as an active ingredient of a PMS-related hot flash-mitigating agent. The hot flash-mitigating agent of the present embodiment can find a wide range of applications, including pharmaceuticals, quasi-drugs, cosmetics, and food and beverage.

The hot flash-mitigating agent of the present embodiment may consist of only a mallow extract or it may be a certain formulation containing a mallow extract.

The hot flash-mitigating agent of the present embodiment can be formulated into a certain formulation (for example, powder, granule, tablet, liquid, etc.) through a conventional formulation method with the use of pharmaceutically acceptable carriers such as dextrin, cyclodextrin and any other auxiliaries. In this case, for example, the auxiliaries include an excipient, a binding agent, a disintegrating agent, a lubricant, a stabilizer, a flavoring agent or corrigent, etc. The hot flash-mitigating agent may be blended with other compositions (for example, external medicine for skin, composition for oral administration, etc.), and may be used as ointment, liquid for external use, adhesive, or the like.

When the hot flash-mitigating agent of the present embodiment is formed into a certain formulation, the content of the mallow extract may not be particularly limited and may be suitably determined according to the purpose.

In addition, in the hot flash-mitigating agent of the present embodiment, an active ingredient thereof may be prepared by blending one or more other natural extracts having a function of mitigating a hot flash with the mallow extract, if necessary.

The hot flash-mitigating agent of the present embodiment may be administered to a patient through transdermal administration, oral administration, and or the like. The administration method may be selected suitably for prevention and treatment of a disease, depending on the type of disease of the patient. Preferably, the transdermal administration may be used. In addition, the dose of the hot flash-mitigating agent of the present embodiment may be suitably increased or decreased depending on the type of disease, severity of disease, individual difference among patients, administration method, administration period, and the like.

The hot flash-mitigating agent of the present embodiment can mitigate hot flashes associated with premenstrual syndrome (PMS) through the hot flash-mitigating action of the mallow extract serving as an active ingredient. The hot flash-mitigating agent according to the present embodiment also can be used in other applications in which it is important to exhibit a hot flash-mitigating effect other than the applications described above.

Since the hot flash-mitigating agent of the present embodiment has a good effect of mitigating a hot flash, it is beneficial to blend the hot flash-mitigating agent of the present embodiment with an external preparation for skin or a composition for oral administration. In this case, as the hot flash-mitigating agent to be blended, a mallow extract may be used as it is, or a certain formulation transformed from the mallow extract may be used.

The external preparation for skin is not particularly limited to a specific classification. Examples of the external preparation for skin include a wide range of quasi-drugs used percutaneously, pharmaceuticals, and the like in addition to cosmetics described later.

The compositions for oral administration are those that are less likely to harm human health and that are ingested by oral or gastrointestinal administration in normal social life. The compositions for oral administration are not constrained to be classified into foods, pharmaceuticals, and quasi-drugs that are categorized according to an administrative classification system. Therefore, term “composition for oral administration” in the present embodiment refers to a wide range of foods, including conventional foods, feeds, health foods, foods with health claims (foods for specified health uses, foods with nutritional claims, and foods with function claims), quasi-drugs, and pharmaceuticals. The composition for oral administration in the present embodiment is preferably an orally administrated composition that can be claimed such that it has beneficial effects provided by a mallow extract on the surface thereof or on the outer packaging thereof. The composition for oral administration in the present embodiment is more preferably a food product with health claims (more specifically, a food product with for specified health uses, a food product with nutrient function claims, or a food product with function claims), a quasi-drug, or a pharmaceutical product.

When the mallow extract is blended into an orally administered composition, the blending ratio of the active ingredient may be suitably determined depending on the purpose of use, symptom, gender, etc. However, considering a normal dosage of intake of the orally administered composition to which the mallow extract is to be added, it is preferable that the daily intake of the mallow extract for an adult is about 1 to 1000 mg. When the orally administered composition to be blended with the mallow extract is in the form of granules, tablets, or capsules, the amount of the mallow extract added is usually 0.1 to 100% by mass and is preferably 5 to 100% by mass, with respect to the orally administered composition to be blended with the mallow extract.

Since the hot flash-mitigating agent of the present embodiment has a beneficial effect of mitigating a hot flash, it can be suitably used as a reagent for research on the action mechanisms thereof.

Since mallow extract has a beneficial effect of improving a hot flash, it is suitably blended into a cosmetic product used to mitigate hot flashes associated with premenstrual syndrome (PMS) (hereinafter, the cosmetic product will be referred to as “hot flash mitigation cosmetic product”). The hot flash-mitigating cosmetic product of the present embodiment can mitigate hot flashes associated with premenstrual syndrome (PMS) through the hot flash-mitigating action of the mallow extract. However, the hot flash mitigation cosmetic product according to the present embodiment can be used in other applications in which it is important to exhibit the effect of mitigating a hot flash as well as the applications described above.

The hot flash mitigation cosmetic product according to the present embodiment may contain mallow extract as it is or may contain a hot flash-mitigating agent formulated from the mallow extract. By blending the mallow extract or the hot flash-mitigating agent described above into cosmetics, it is possible to impart a hot flash-mitigating effect to cosmetics.

The hot flash mitigation cosmetic product of the present embodiment is not particularly limited to a specific cosmetic product, and the examples of the cosmetic product include essences, gels, creams, milky lotions, emulsions, all-in-one cosmetics, lotions, facial masks, foundations, and lip balms. Among them, to mitigate hot flashes and to moisturize the skin so that the skin becomes soft and elastic, the hot flash mitigation cosmetic product of the present invention is preferably implemented as a cosmetic essence. Alternatively, to supply both watery and oily moisturizing ingredients to the skin to strengthen the barrier function of the skin and to protect the skin from external irritation as well as to mitigate a hot flash, the hot flash mitigation cosmetic product of the present invention is preferably implemented as a gel preparation.

The hot flash mitigation cosmetic product of the present embodiment may be implemented as a one-step cosmetic such as an all-in-one cosmetic containing a mallow extract. Alternatively, the cosmetic product may be composed of a first cosmetic product implemented as a cosmetic essence containing a mallow extract and a second cosmetic product implemented as a gel preparation containing a mallow extract. When the cosmetic product is composed of the first cosmetic product and a second cosmetic product, it is preferable that the first cosmetic product and the second cosmetic product are contained in respective containers.

When the mallow extract or the hot flash-mitigating agent is blended into a hot flash mitigation cosmetic product, the blending ratio may be suitably adjusted depending on the type of the hot flash mitigation cosmetic product. For example, a preferable blending ratio is 0.0001 to 5% by mass (in terms of solid content), and a more preferable blending ratio is 0.0001 to 0.01% by mass (in terms of solid content).

The hot flash mitigation cosmetic product of the present embodiment may use a main component that is used in the production of ordinary cosmetics, in combination with an auxiliary component or other components such as an astringent agent, a bactericidal/antibacterial agent, an UV absorber, a moisturizer, a cell activator, an anti-inflammatory/anti-allergic agent, an antioxidant/active oxygen remover, fats and oils, waxes, hydrocarbons, fatty acids, alcohols, esters, surfactants, perfumes and the like if the auxiliary components or other components do not interfere with the hot flash-mitigating effect provided by the mallow extract. When the components are collaboratively used, the cosmetic product becomes a more common product, and a superior synergistic effect to what is normally expected can be obtained.

When the cosmetic product of the present embodiment is composed of the first cosmetic product and the second cosmetic product, the first cosmetic product necessarily contains mallow extract as an active ingredient and optionally contains other ingredients such as purified water, dipropylene glycol, butylene glycol, glycerin, and the like, if necessary. The second cosmetic product necessarily contains mallow extract as an active ingredient and optionally contains other ingredients such as purified water, glycerin, an acrylic alkyl methacrylate copolymer, sodium hydroxide, and the like, if necessary.

The hot flash mitigation cosmetic product of the present embodiment may be used in a single step, like an all-in-one cosmetic product, or may be used in two sequential steps in which the first cosmetic product and the second cosmetic product are used in respective steps. Therefore, the hot flash mitigation cosmetic product of the present embodiment can be conveniently used according to the daily makeup behavior of the user. In addition, the user can wear the cosmetic product without feeling confused when using it. When the cosmetic product is used in two steps, one of the first cosmetic product and the second cosmetic product may be used first, and the other may be used later. It is preferable to use the first cosmetic product and the second cosmetic product in this order.

In addition, when using the hot flash mitigation cosmetic product of the preset embodiment, in the case where the first cosmetic product and the second cosmetic product are in sequential two steps, the effects of the respective cosmetic products can be effectively exhibited. For example, when the second cosmetic product is a gel preparation, the effect of mitigating a hot flash can be obtained by the action of the active ingredient, and the moisturizing effect given by the first cosmetic product can be preserved.

The application dose of the hot flash mitigation cosmetic product of the present embodiment may be suitably increased or decreased depending on individual preference, using method, time of use, and period of use. The time of use is preferably a time prior to the scheduled menstruation and more preferably 9 to 5 consecutive days before the scheduled menstrual period. The period of use is preferably 2 to 10 consecutive days and more preferably 4 to 8 consecutive days.

### Example

Hereinafter, the present invention will be described in greater detail with reference to test examples, but the present invention is not limited to the examples described below.

### Preparation Example 11 Preparation of Mallow Extract

4 kg of 50% BG (1,3 butylene glycol, high sugar cane BG, manufactured by Kokyu Alcohol Kogyo Co., Ltd.) was mixed with 20 g of dried flowers, leaves, and stems of mallow (Malva mauritiana L) and was immersed for 7 days. The resulting product was filtered with ADVANTEC qualitative filter paper (No. manufactured by Toyo Roshi Kaisha Ltd.) and a membrane (0.45 μm) to obtain filtrate serving as a mallow extract (solid content: 0.15% by mass).

### [Test Example 1] Assessment of Efficacy on Hot Flashes

Essences and gels listed below were prepared.

[Preparation of Essence A]

| | |
|---|---|
| Purified water | 74.96 g |
| Dipropylene glycol | 10.0 g |
| 1,3-butylene glycol | 7.0 g |
| Concentrated glycerin | 6.0 g |
| Diglycerin | 1.0 g |
| Mallow extract (Preparation Example 1) | 0.4 g |
| Phenoxyethanol | 0.4 g |
| Carbomer | 0.2 g |
| Sodium hydroxide | 0.04 g |

[Preparation of Essence B]

| | |
|---|---|
| Purified water | 75.36 g |
| Dipropylene glycol | 10.0 g |
| 1,3-butylene glycol | 7.0 g |
| Concentrated glycerin | 6.0 g |
| Diglycerol | 1.0 g |
| Phenoxyethanol | 0.4 g |
| Carbomer | 0.2 g |
| Sodium hydroxide | 0.04 g |

[Preparation of Gel A]

| | |
|---|---|
| Purified water | 85.55 g |
| 1,3-butylene glycol | 7.0 g |
| Concentrated glycerin | 5.0 g |
| Diglycerol | 1.0 g |
| Acrylic acid-alkyl methacrylate copolymer | 0.5 g |
| Phenoxyethanol | 0.4 g |
| Mallow extract (Preparation Example 1) | 0.4 g |
| Sodium hydroxide | 0.15 g |

[Preparation of Gel B]

| | |
|---|---|
| Purified water | 85.95 g |
| 1,3-butylene glycol | 7.0 g |
| Concentrated glycerin | 5.0 g |
| Diglycerol | 1.0 g |
| Acrylic acid-alkyl methacrylate copolymer | 0.5 g |
| Phenoxyethanol | 0.4 g |
| Sodium hydroxide | 0.15 g |

The improvement effects of Essence A, Essence B, Gel A, and Gel B that were prepared as described above on hot flashes were assessed. In addition, visual analog scale (VAS), which is a self-rating scale, was used to assess the feeling of a hot flash.

For five days starting from the seventh day prior to the start of a menstrual period, four women in their 30s to 40s who usually feel a hot flash before their menstruation used their usual detergent, facial cleanser, and location as usual and then sequentially applied Essence A and Gel A on their left half face (Example 1). The four women sequentially applied Essence B and Gel B on their right half face (Comparative Example 1).

After 5 days of use, a VAS questionnaire survey (10 for a case of feeling strong hot flashes, and 0 for a case of feeling no hot flashes at all) was performed to measure normal premenstrual warmth (when a usual cleansing agent, facial cleanser, skin lotion, essence, and gel were used) as Comparative Example 2. The results are shown in Tables 1 and 2.

TABLE 1

| | Comparative Example 2 | Comparative Example 1 |
|---|---|---|
| Age | (left half face) | (left half face) |
| 35 | 3.9 | 0.5 |
| 33 | 1.8 | 0 |
| 41 | 1.0 | 0.1 |
| 32 | 4.7 | 0 |
| Average | 3.1 | 0.2 |

| | |
|---|---|
| t-test | 0.025 |

TABLE 2

| | Comparative Example 2 | Comparative Example 1 |
|---|---|---|
| Age | (right half face) | (right half face) |
| 35 | 1.8 | 1.8 |
| 33 | 0.2 | 0 |
| 41 | 0.4 | 0.1 |
| 32 | 5.0 | 0 |
| Average | 1.9 | 0.5 |

| | |
|---|---|
| t-test | 0.338 |

As shown in Table 1, the average VAS of Comparative Example 2 (left half face) was 3.1, whereas the average VAS of Example 1 was 0.2 which was significantly lower than that of Comparative Example 2. That is, it was confirmed that hot flashes associated with premenstrual syndrome (PMS) were mitigated. On the other hand, as shown in Table 2, the average VAS of Comparative Example 2 (right half face) was 1.9, whereas the average VAS of Comparative Example 1 was 0.5. That is, no significant difference was observed.

[Blending Example 1]

A cream having the following composition was produced by a conventional method:

| | |
|---|---|
| Mallow extract (Preparation Example 1) | 0.5 g |
| Sophora extract | 0.1. g |
| Scutellaria root extract | 0.1 g |
| Liquid paraffin | 5.0 g |
| White beeswax | 4.0 g |
| Squalane | 10.0 g |
| Cetanol | 3.0 g |
| Lanolin | 2.0 g |
| Stearic acid | 1.0 g |
| Polyoxyethylene Sorbitan Monooleate (20E.O.) | 1.5 g |
| Glyceryl monostearate | 3.0 g |
| Licorice extract (glycyrrhiza extract) | 0.1 g |
| 1,3-butylene glycol | 6.0 g |
| Methyl Para hydroxybenzoate | 1.5 g |
| Perfume | 0.1 g |
| Purified water | the remainder (based on |
| | a total amount of 100 g) |

[Blending Example 2]

A milky lotion having the following composition was prepared by a conventional method:

| | |
|---|---|
| Mallow extract (Preparation Example 1) | 1.0 g |
| Jojoba oil | 4.00 g |
| 1,3-butylene glycol | 3.00 g |
| Arbutin | 3.00 g |
| Polyoxyethylene cetyl ether (20E.O.) | 2.50 g |
| Olive oil | 2.00 g |
| Squalane | 2.00 g |
| Cetanol | 2.00 g |
| Glyceryl monostearate | 2.00 g |
| Polyoxyethylene Sorbitan Monooleate (20E.O.) | 2.0 g |
| Methyl parahydroxybenzoate | 0.15 g |
| Stearyl glycyrrhizate | 0.10 g |
| Engelhardtia roxburghiana extract | 0.10 g |
| Dipotassium glycyrrhizinate | 0.10 g |
| Ginkgo leaf extract | 0.10 g |
| Conchiolin | 0.10 g |
| Cork tree bark extract | 0.10 g |
| Chamomilla recutita extract | 0.10 g |
| Perfume | 0.05 g |
| Purified water | the remainder (based on |
| | a total amount of 100 g) |

[Blending Example 3]

A beauty essence having the following composition was produced by a conventional method:

| | |
|---|---|
| Mallow extract (Preparation Example 1) | 0.05 g |
| Chamomile extract | 0.1 g |
| Carrot extract | 0.1 g |
| Xanthan gum | 0.3 g |
| Hydroxyethyl cellulose | 0.1 g |
| Carboxyvinyl polymer | 0.1 g |
| 1,3-butylene glycol | 4.0 g |
| Dipotassium glycyrrhizinate | 0.1 g |
| Glycerin | 2.0 g |
| Potassium hydroxide | 0.25 g |
| Fragrance | 0.01 g |
| Preservative (methyl paraoxybenzoate) | 0.15 g |
| Ethanol | 2.0 g |
| Purified water | the remainder (based on |
| | a total amount of 100 g) |

The hot flash-mitigating agent of the present invention can considerably contribute to the mitigation of hot flashes associated with premenstrual syndrome (PMS).

What is claimed is:

1. An agent for mitigating hot flashes associated with premenstrual syndrome (PMS), the agent containing mallow extract as an active ingredient.

2. A cosmetic product used to mitigate hot flashes associated with premenstrual syndrome (PMS), the cosmetic product containing mallow extract.

3. The cosmetic product according to claim 2, comprising:
to a first cosmetic product that is an essence into which mallow extract is blended; and
a second cosmetic product that is a gel preparation into which mallow extract is blended,
wherein the first cosmetic product and the second cosmetic product are contained in respective containers.

4. A method of using the cosmetic product according to claim 3, wherein the first cosmetic product and the second cosmetic product are sequentially applied.
